(19) United States
(12) Patent Application Publication
(10) Pub. No.: US 2009/0011025 A1
(43) Pub. Date: Jan. 8, 2009

(54) Biotin-Amino Acid Conjugate Useful as a Hydrogelator and Hydrogel Prepared Therefrom

(75) Inventors: Byeang Hyean Kim, Kyungsangbuk-do (KR); Sankaprasad Bhuniya, Kyungsangbuk-do (KR); Sun Min Park, Kyungsangbuk-do (KR)

Correspondence Address: SUGHRUE MION, PLLC, 2100 PENNSYLVANIA AVENUE, N.W., SUITE 800, WASHINGTON, DC, 20037, US

(73) Assignees: POSTECH FOUNDATION, POHANG-SI (KR); POSTECH ACADEMY-INDUSTRY FOUNDATION, POHANG-SI (KR)

(21) Appl. No.: 11/913,856

(22) PCT Filed: Sep. 23, 2005
(86) PCT No.: PCT/KR05/03156

§ 371 Date: Nov. 8, 2007

(30) Foreign Application Priority Data
May 12, 2005 (KR) 10-2005-0039892

(51) Int. Cl. C07D 495/04; A61K 9/10

(52) U.S. Cl. 424/486; 548/304.1

(57) ABSTRACT

A biotin-amino acid conjugate, wherein the carboxylic group of biotin and the α-amino group of an amino acid is linked by an amide bond, is biocompatible and has an excellent gelation capability in an aqueous medium. Accordingly, a hydrogel prepared therefrom is useful as a drug delivery system.

### FIELD OF THE INVENTION

The present invention relates to a biotin-amino acid conjugate useful as a hydrogelator for preparing a thermostable and biocompatible hydrogel, and a drug delivery system prepared therefrom.

### BACKGROUND OF THE INVENTION

Although very widespread in nature, for instance, in the growth of animal cells, gels are becoming advanced materials for high-technology applications in such fields as drug delivery (Okano, T., Biorelated Polymers and Gels, Academic Press, San Diego, 1998) and tissue engineering, and as scaffolds (Dagani, R., Chem. Eng. News 1997, 75, 26; Nishikawa, T. et al., J. Am. Chem. Soc. 1996, 118, 6110; and Osada, Y and Gong, J. P., Adv. Mater 1998, 10, 827).

Recently, supramolecular self-assembly approaches have been used to prepare hydrogels from low-molecular-weight compounds, such as simple amphiphiles (Menger, F. M. et al., J. Am. Chem. Soc. 2002, 124, 1140), bolaamphiphiles (Acharya, S, N. G., Chem. Mater. 1999, 11, 3504.), gemini surfactants (Iwaura, R. et al., Angew. Chem., Int. Ed. 2003, 42, 1009) and other hydrogelators (Yang, Z. et al., Chem. Commun. 2004, 208; and Numata, K. M. et al., Chem. Commun. 2004, 1996). The supramolecular hydrogels are formed when the monomer units self-assemble into polymer-like fibers that immobilize solvent molecules. A similar approach in which drugs or vitamins are used directly to form hydrogels has been suggested to lead to new types of biomaterials that may function as “self-delivery” systems (Xing, B. et al., J. Am. Chem. Soc. 2002, 124, 14846).

Meanwhile, biotin (vitamin H) has a clinical significance due to its abilities for helping the synthesis of fatty acid and oxidation of fatty acid and carbohydrate, and enhancing the bioavailability of a protein, folic acid, panthothenic acid and vitamin B₁₂ (Friedrich, W., Vitamins, Walter de Grueter & Co, Berlin, 1998). Because of its low solubility in water (1.0-0.8 mmol/L) and insolubility in organic solvents, however, biotin has a low bioavailability that severely restricts its effectiveness and, therefore, it has hitherto been used only to very limited purposes.

Although biotin-based organogel has been reported (Crisp, G. T. and Gore, J., Syn. Commun. 1997, 27, 2203), it can not be employed as a drug delivery system because it is not biocompatible.

The present inventors have endeavored to develop a hydrogelator useful as a material for drug delivery, and have discovered that a low-molecular weight biotin-amino acid conjugate is suitable for in vivo applications and exhibit a remarkable gelation properties in an aqueous medium, and, therefore, a hydrogel prepared therefrom is useful as a drug delivery system.

### SUMMARY OF THE INVENTION

Accordingly, it is an object of the present invention to provide a gelator compound having remarkable gelation properties in aqueous media, and a process for the preparation thereof.

It is another object of the present invention to provide a hydrogel prepared by employing the gelator compound.

It is a further object of the present invention to provide a drug delivery system comprising the hydrogel.

In accordance with one aspect of the present invention, there is provided a biotin-amino acid conjugate, wherein the carboxylic group of biotin and the α-amino group of the amino acid is linked by an amide bond.

In accordance with another aspect of the present invention, there is provided a hydrogel prepared by dissolving the biotin-amino acid conjugate in an aqueous medium.

In accordance with another aspect of the present invention, there is provided a drug delivery system comprising the hydrogel and a drug incorporated therein.

### BRIEF DESCRIPTION OF THE DRAWINGS

The above and other objects and features of the present invention will become apparent from the following description of the invention, when taken in conjunction with the accompanying drawings, which respectively show:

FIG. 1: scanning electron microscopy (SEM) images of the xerogels of gelators 1, 2, 4, 5 and 8 to 11.

FIG. 2: FT-IR spectra of gelators 5, 9 and 11 in their solid (solid line) and gel (dotted line) states.

FIG. 3: changes in the proton chemical shifts of the ureido and amide moieties of the gelators in solutions containing various ratios (v/v) of DMSO-d₆ and H₂O.

FIG. 4: theoretical assessments of the effective hydrogen bondings of the dimer of gelator 5 using MOPAC6 modeling.

FIG. 5: SEM images of the hydrogels prepared from gelator 9 before (a) and after (b) the addition of streptavidin.

FIG. 6A: the concentration of zidobudine (AZT) released from AZT/gelator 9 hydrogel and AZT/gelator 9 hydrogel with streptavidin over time.

FIG. 6B: SEM images of the hydrogels of gelator 9 and AZT/gelator 9.

### DETAILED DESCRIPTION OF THE INVENTION

The preferred biotin-amino acid conjugates of the present invention are represented by formula (I):

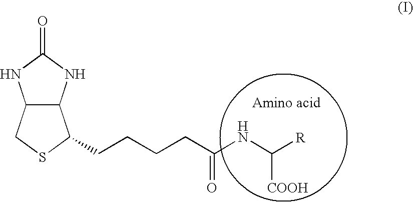

wherein, R is C₁-C₆ alkyl, or C₁-C₃ alkyl substituted with phenyl, methyl, methylthio, hydroxyphenyl or indole.

The most preferred biotin-amino acid conjugates of the present invention are those of formula (I), wherein R is C₄-C₆ alkyl or phenylmethyl.

The inventive biotin-amino acid conjugate has a free carboxyl group, like biotin itself, and exhibits variable hydrophobicity depending on the kind of the amino acid moiety. Further, the biotin-amino acid conjugate retains the receptor binding site, i.e., unaltered ureido moiety, and, accordingly, it can form receptor-ligand interactions with suitable receptors such as avidin, streptavidin, cyclodextrin and insulin.

As shown in the following Reaction Scheme I, the inventive biotin-amino acid conjugate can be prepared by forming a new amide bond between the carboxylic group of biotin and the α-amino group of an amino acid.

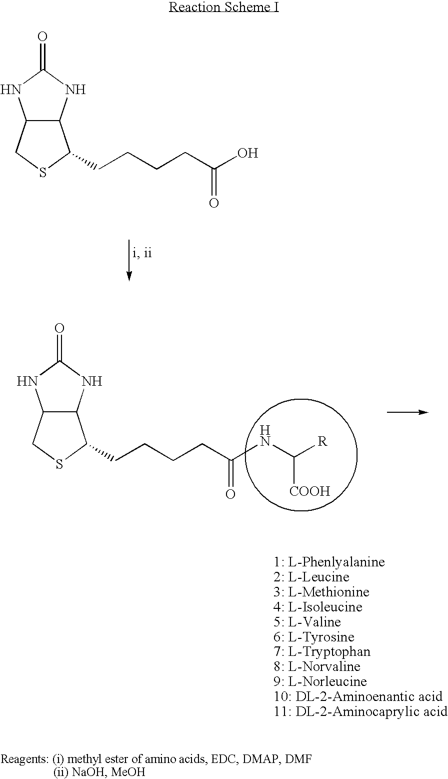

Specifically, D-biotin, 1-(3-dimethylaminopropyl)-3-ethylcarbodiimide.HCl (EDC) and N,N-dimethyl-4-aminopyridine (DMAP) are dissolved in a suitable organic solvent such as dimethylformamide (DMF), dichloromethane, trichloromethane and tetrahydrofuran, and a methyl ester of an amino acid is added thereto, and the mixture is allowed to react for 2 to 10 hours, preferably, for 4 to 6 hours, to obtain an intermediate compound. The intermediate compound is treated with NaOH in a mixture of an organic solvent and water, e.g., a mixture of methanol and water, for 2 to 10 hours, preferably, 4 to 6 hours to obtain the biotin-amino acid conjugate. In the above preparation process, the respective reactions are preferably carried out at room temperature.

The inventive biotin-amino acid conjugate (“gelator”) forms a hydrogel upon dissolution in an aqueous medium such as water, saline and various buffers having a wide range of pH. The hydrogel texture, which reflects their stabilities, may vary widely depending on the properties of side chains on their amino acid moieties, and the xerogels formed from the inventive hydrogels show two types of gels, fibrous and lamella. Generally, the hydrogels formed by gelators having a long linear alkyl chain on the amino acid moiety exhibit fibrous structures, wherein the diameter of the fibers ranging from 20 to 50 nm. On the other hand, the hydrogels formed by gelators having a branched or short alkyl chain on the amino acid moiety show significantly thick lamellar structures.

Further, the nature of the hydrophobic residue on the amino acid moiety of the gelator has a great influence on the stability and clarity of the gel formed by the gelator. Specifically, the gelators having a long linear alkyl chain form very stable gels that persisted for about 6 months in aqueous media, and the clarities of the gels ranged from translucent to opaque. Meanwhile, hydrogels formed by gelators having a branched or short alkyl chain are relatively unstable and opaque.

Moreover, the minimum gelation concentration (MGC) values of the inventive hydrogels measured in 0.9% NaCl solution are equivalent to those measured in distilled water, which means that the inventive biotin-amino acid conjugate can be used for in vivo applications.

A drug delivery system wherein a drug is incorporated in a hydrogel can be obtained by dissolving the inventive biotin-amino acid conjugate in an aqueous medium to form a hydrogel and adding the drug thereto. The drug delivery system thus prepared slowly releases the drug incorporated in the hydrogel and, accordingly, the inventive biotin-amino acid conjugate is very useful as a biomaterial for the preparation of a drug delivery system.

Meanwhile, in the inventive biotin-amino acid conjugate, the ureido group of the biotin moiety forms an intermolecular hydrogen bonding to the terminal carboxyl group of other gelator molecule, which leads to the formation of a hydrogel as a self-assembled polymer chain. When a receptor of biotin such as avidin, streptavidin, cyclodextrin and insulin specifically bind to the ureido group of biotin by the ligand-receptor interaction, the fiber network of the biotin-based gelator becomes disrupted, resulting in faster release of the drug. Accordingly, in preparing a drug delivery system, it is advantageous to add a biotin receptor to the medium containing the hydrogel of biotin-amino acid conjugate, so that the drug release rate can be controlled by the amount of the biotin receptor.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### REFERENCE EXAMPLE

### Materials and Instrumental Analyses

All chemicals used in the following Examples were obtained from Aldrich Chemical Company and used without further purification. Each reaction was executed under an inert atmosphere of dry argon using glassware that was flame-dried under vacuum. Flash chromatography was performed using silica gel 60 (230-400 mesh; ASTM). Melting points were obtained using an Electrothermnal 1A 9000 series apparatus. FT-IR spectra were recorded on a Brucker model FT-IR PS55+ spectrometer. Low-resolution FAB⁺ mass spectra were obtained using a JEOL JMS-AX505WA (FAB) spectrometer. ¹H and ¹³C NMR spectra were recorded using a Bruker Aspect 300 NMR spectrometer. Chemical shifts were reported in parts per million (ppm) downfield relative to the internal standard, tetramethylsilane (TMS). Coupling constants were reported in hertz (Hz). Spectral splitting patterns were designated as s, singlet; d, doublet; dd, double doublet; dt, distorted triplet; t, triplet; m, multiplet; and br, broad. SEM images were obtained using a Philips XL30S FEG SEM analyzer.

### EXAMPLE

### Synthesis of Biotin-Amino Acid Conjugate Hydrogelators

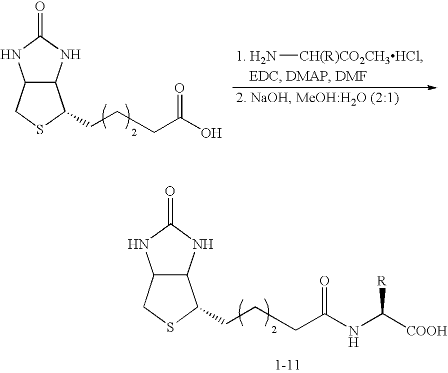

### Example 1

### Preparation of N-biotinyl-L-phenylalanine (Gelator 1)

### (Step 1)

D-biotin (244 mg, 0.1 mmol), 1-(3-dimethylaminopropyl)-3-ethylcarbodiimide.HCl (EDC; 192 mg, 0.1 mmol), and N,N-dimethyl-4-aminopyridine (DMAP; 122 mg, 0.1 mmol) were dissolved in dry DMF (25 ml) and placed in a two-neck round-bottom flask equipped with an argon gas inlet. L-phenylalanine methyl ester hydrochloride (115 mg, 0.1 mmol) was added thereto, and the mixture was stirred for 4 hours. When the reaction was completed, the mixture was poured into water and extracted with CH₂Cl₂. The product was isolated and purified by column chromatography (SiO₂; CH₂Cl₂/MeOH, 10:1) to obtain 309 mg of compound 1a (yield: 76%).

### (Step 2)

Compound 1a (202 mg, 0.5 mmol) was placed in a 25-ml round-bottom flask, and then distilled water (4 ml) and MeOH (8 ml) were added thereto. NaOH (60 mg, 3 equiv.) was added to the flask and the mixture was stirred at room temperature for 4 hours. After the reaction was completed, the mixture was acidified using dilute sulfuric acid to pH 2-3. The hydrolyzed product that precipitated was filtered, dried in the air, and then washed several times with acetone and CH₂Cl₂. The product was isolated by column chromatography (SiO₂; toluene/MeOH/acetone/AcOH, 14:4:1:1) and then freeze-dried to yield gelator compound 1 (195 mg).

M.p. 205° C.

¹H NMR (300 MHz, DMSO-d₆): 8.12 (d, J=7.9 Hz, 1H; NH), 7.30-7.20 (m, 5H; Ar—H), 6.41 (s, 1H; N³H), 6.10 (s, 1H; N¹H), 4.43 (dt, J₁=7.1 Hz, J₂=3.5 Hz, 1H, CH), 4.31 (dd, J₁=7.1 Hz, J₂=5.0 Hz, 1H; CH), 4.12 (dd, J₁=6.3 Hz, J₂=4.4 Hz, 1H; CH), 3.07 (dd, J₁=8.8 Hz, J₂=4.9 Hz, 1H; CH), 2.87 (d, J=5.1 Hz, 2H; CH₂), 2.80 (dd, J=4.8 Hz, J_{gem}=12.0 Hz, 1H; CH₂), 2.56 (d, J_{gem}=12.0 Hz, 1H; CH₂), 2.05 (t, J=6.9 Hz, 2H; CH₂), 1.44-1.40 (br, 4H; CH₂), 1.26-1.19 (m, 2H; CH₂).

¹³C NMR (75.5 MHz, DMSO-d₆): 173.35, 172.16, 162.79, 137.82, 129.11, 128.19, 126.41, 61.04, 59.22, 55.49, 53.33, 38.95, 38.67, 36.78, 34.86, 28.01, 25.24.

MS (FAB): m/z 392 [M+H]⁺.

Anal. Calcd. for C₁₉H₂₅N₃O₄S: C, 58.28; H, 6.44; N, 10.73; S, 8.19. Found: C, 57.89; H, 6.43; N, 10.70; S, 8.13.

### Example 2

### Preparation of N-biotinyl-L-leucine (Gelator 2)

The procedure of Step 1 of Example 1 was repeated, except for employing L-leucine methyl ester hydrochloride (182 mg, 0.1 mmol) in place of L-phenylalanine methyl ester hydrochloride, to obtain compound 2a (309 mg, yield: 81%). Further, employing compound 2a (186 mg), the procedure of Step 2 of Example 1 was repeated to obtain gelator compound 2 (178 mg).

M.p. 209-210° C.

¹H NMR (300 MHz, DMSO-d₆): 8.03 (d, J=7.56 Hz. 1H; NH), 6.43 (s, 1H; N³H), 6.38 (s, 1H; N¹H), 4.31 (dt, J₁=6.5 Hz, J₂=5.0 Hz, 1H; CH), 4.29 (dd, J₁=8.4 Hz, J₂=4.8 Hz, 1H; CH), 4.13 (dd, J₁=5.6 Hz, J₂=4.3 Hz, 1H; CH), 3.08 (dt, J₁=7.2 Hz, J₂=5.0 Hz, 1H; CH), 2.84 (dd, J=4.2 Hz, J_{gem}=12.9 Hz, 1H; CH₂), 2.62 (d, J_{gem}=12.9 Hz, 1H; CH₂), 2.12 (t, J=6.0 Hz, 2H; CH₂), 1.66-1.48 (br, 6H; CH₂), 1.35 (m, 2H; CH₂), 1.12 (m, 1H; CH), 0.88 (d, J=3.4 Hz, 6H; CH₃).

¹³C NMR (75.5 MHz, DMSO-d₆): 173.91, 171.82, 162.36, 60.71, 58.89, 55.07, 49.71, 40.06, 38.39, 34.46, 27.68, 27.63, 24.88, 24.00, 22.50, 20.92.

MS (FAB): m/z 358 [M+H]⁺.

Anal. Calcd. for C₁₆H₂₇N₃O₄S: C, 53.76; H, 7.61; N, 11.73; S, 8.97. Found: C, 53.56; H, 7.77; N, 11.55; S, 8.87.

### Example 3

### Preparation of N-biotinyl-L-methionine (Gelator 3)

The procedure of Step 1 of Example 1 was repeated, except for employing L-methionine methyl ester hydrochloride (200 mg, 0.1 mmol) in place of L-phenylalanine methyl ester hydrochloride, to obtain compound 3a (300 mg, yield: 72%). Further, employing compound 3a (195 mg), the procedure of Step 2 of Example 1 was repeated to obtain gelator compound 3 (188 mg).

M.p. 207° C.

¹H NMR (300 MHz, DMSO-d₆): 8.11 (d, J=7.8 Hz, 1H; NH), 6.43 (s, 1H; N³H), 6.34 (s, 1H; N¹H), 4.31 (dt, J₁=7.5 Hz, J₂=4.5 Hz, 1H; CH), 4.23 (dd, J₁=7.1 Hz, J₂=4.2 Hz, 1H; CH), 4.12 (dd, J₁=6.9 Hz, J₂=4.3 Hz, 1H; CH), 3.06 (dt, J₁=7.2 Hz, J₂=4.4 Hz, 1H; CH), 2.79 (dd, J=4.5 Hz, J_{gem}=13.5 Hz, 1H; CH₂), 2.50 (d, J_{gem}=13.5 Hz, 1H; CH₂), 2.45 (t, J=6.8 Hz, 2H; CH₂), 2.13 (t, J=6.0 Hz, 2H; CH₂), 2.09 (s, 3H; CH₃), 1.88-1.76 (m, 2H; CH₂), 1.50-1.48 (br, 4H; CH₂), 1.32 (m, 2H; CH₂).

¹³C NMR (75.5 MHz, DMSO-d₆): 173.51, 172.34, 162.79, 61.00, 59.10, 55.42, 50.77, 40.28, 34.75, 30.51, 29.72, 28.93, 27.9, 25.71, 14.57.

MS (FAB): m/z 376 [M+H]⁺.

Anal. Calcd. for C₁₅H₂₅N₃O₄S₂: C, 47.98; H, 6.71; N, 11.19; S, 17.08. Found: C, 47.77; H, 6.55; N, 10.97; S, 17.12.

### Example 4

### Preparation of N-biotinyl-L-isoleucine (Gelator 4)

The procedure of Step 1 of Example 1 was repeated, except for employing L-isoleucine methyl ester hydrochloride (182 mg, 0.1 mmol) in place of L-phenylalanine methyl ester hydrochloride, to obtain compound 4a (246 mg, yield: 66%). Further, employing compound 4a (186 mg), the procedure of Step 2 of Example 1 was repeated to obtain gelator compound 4 (178 mg).

M.p. 230-233° C.

¹H NMR (300 MHz, DMSO-d₆): 7.95 (d, J=8.5 Hz, 1H; NH), 6.40 (s, 1H; N³H), 6.34 (s, 1H; N¹H), 4.27 (dt, J₁=7.2 Hz, J₂=5.2 Hz, 1H; CH), 4.14 (dd, J₁=7.2 Hz, J₂=4.1 Hz, 1H; CH), 4.07 (dd, J₁=7.8 Hz, J₂=5.6 Hz, 1H; CH), 3.03 (dt, J₁=6.3 Hz, J₂=4.3 Hz, 1H; CH), 2.75 (dd, J=6.0 Hz, J_{gem}=13.2 Hz, 1H; CH₂), 2.50 (d, J_{gem}=13.2 Hz, 1H; CH₂), 2.12 (t, J=6.0 Hz, 2H; CH₂), 1.52-1.48 (m, 1H; CH), 1.44-1.35 (br, 4H; CH₂), 1.17-1.12 (m, 2H; CH₂), 0.80-0.76 (m, 6H; CH₃).

¹³C NMR (75.5 MHz, DMSO-d₆): 172.77, 171.81, 162.21, 60.52, 58.66, 55.58, 38.10, 35.56, 34.16, 27.59, 27.47, 24.83, 24.18, 15.01, 10.71

MS (FAB): m/z 358 [M+H]⁺.

Anal. Calcd. for C₁₆H₂₇N₃O₄S: C, 53.76; H, 7.61; N, 11.73; S, 8.97. Found: C, 53.62; H, 7.57; N, 11.59; S, 8.95.

### Example 5

### Preparation of N-biotinyl-L-valine (Gelator 5)

The procedure of Step 1 of Example 1 was repeated, except for employing L-valine methyl ester hydrochloride (167 mg, 0.1 mmol) in place of L-phenylalanine methyl ester hydrochloride and a mixture of CH₂Cl₂/MeOH (9:1) as an eluant in the column chromatography, to obtain compound 5a (165 mg, yield: 50%). Further, employing compound 5a (179 mg), the procedure of Step 2 of Example 1 was repeated to obtain gelator compound 5 (156 mg, yield: 91%).

M.p. 215-216° C.

¹H NMR (300 MHz, DMSO-d₆): 7.80 (d, J=8.7 Hz, 1H; NH), 6.36 (s, 1H; N³H), 6.30 (s, 1H; N¹H), 4.27 (dd, J₁=7.2 Hz, J₂=4.9 Hz, 1H; CH), 4.13 (dd, J₁=5.8 Hz, J₂=3.2 Hz, 1H; CH), 4.00 (dd, J₁=6.0 Hz, J₂=3.2 Hz, 1H; CH), 3.00 (dt, J₁=6.4 Hz, J₂=4.4 Hz, 1H; CH), 2.75 (dd, J=3.5 Hz, J=13.2 Hz, 1H; CH₂), 2.45 (d, J=13.2 Hz, 1H; CH₂), 2.09 (t, J=6.0 Hz, 2H; CH₂), 1.92 (m, 1H; CH), 1.53-1.41 (br, 4H; CH₂), 1.23-1.20 (m, 2H; CH₂), 0.78 (d, J=6.7 Hz, 6H; CH₃).

¹³C NMR (75.5 MHz, DMSO-d₆): 172.50, 171.67, 161.96, 60.26, 58.40, 56.24, 54.67, 37.84, 33.91, 28.94, 27.35, 27.22, 24.59, 18.41, 17.29.

MS (FAB): m/z 343.94 [M+H]⁺.

Anal. Calcd. for C₁₆H₂₇N₃O₄S: C, 52.46; H, 7.34; N, 12.23; S, 9.04. Found: C, 52.37; H, 7.46; N, 11.92; S, 8.79.

### Example 6

### Preparation of N-biotinyl-L-tyrosine (Gelator 6)

The procedure of Step 1 of Example 1 was repeated, except for employing L-tyrosine methyl ester hydrochloride (231 mg, 0.1 mmol) in place of L-phenylalanine methyl ester hydrochloride, to obtain compound 6a (212 mg, yield: 50%). Further, the procedure of Step 2 of Example 1 was repeated, employing compound 6a (210 mg) and a mixture of CH₂Cl₂/MeOH/acetone/AcOH (14:4:1:1) as an eluant in the column chromatography, to obtain gelator compound 6 (177 mg, yield: 87%).

M.p. 259-260° C.

¹H NMR (300 MHz, DMSO-d₆): 9.22 (s, 1H; ArOH), 8.09 (d, J=8.1 Hz, 1H; NH), 7.00 (d, J=8.1, 2H; ArH), 6.60 (d, J=8.0 Hz, 2H; ArH), 6.44 (s, 1H; N³H), 6.40 (s, 1H; N¹H), 4.32 (dt, J₁=6.5 Hz, J₂=4.1 Hz, 1H; CH), 4.28 (dd, J=7.2 Hz, J₂=4.8 Hz, 1H; CH), 4.11 (dd, J₁=7.0 Hz, J₂=5.1 Hz, 1H; CH), 3.04 (dt, J₁=6.6 Hz, J₂=4.1 Hz, 1H; CH), 2.92 (d, J=7.0 Hz, 2H; CH₂), 2.75 (dd, J=6.0 Hz, J_{gem}=12.3 Hz, 1H; CH₂), 2.59 (d, J_{gem}=12.3 Hz, 1H; CH₂), 2.08 (t, J=6.3 Hz, 2H; CH₂), 1.54-1.40 (br, 4H; CH₂), 1.22-1.20 (m, 2H; CH₂).

¹³C NMR (75.5 MHz, DMSO-d₆): 173.29, 172.07, 162.70, 155.83, 129.93, 127.74, 114.93, 61.01, 59.22, 55.37, 53.58, 40.39, 36.04, 34.86, 27.95, 25.14.

MS (FAB): m/z 407.99 [M+H]⁺.

Anal. Calcd. for C₁₉H₂₅N₃O₅S: C, 56.00; H, 6.18; N, 10.31; S, 7.87. Found: C, 55.85; H, 6.00; N, 10.42; S, 7.87.

### Example 7

### Preparation of N-biotinyl-L-tryptophan (Gelator 7)

The procedure of Step 1 of Example 1 was repeated, except for employing L-tryptophan methyl ester hydrochloride (254 mg, 0.1 mmol) in place of L-phenylalanine methyl ester hydrochloride, increased reaction time of 6 hours, and a mixture of CH₂Cl₂/MeOH (12:1) as an eluant in the column chromatography, to obtain compound 7a (365 mg, yield: 82%). Further, employing compound 7a (222 mg, 0.05 mmol), the procedure of Step 2 of Example 1 was repeated to obtain gelator compound 7 (215 mg).

M.p. 160-161° C.

¹H NMR (300 MHz, DMSO-d₆): 8.14 (d, J=7.8 Hz, 1H; NH), 7.55 (d, J=6.9 Hz, 1H; NH), 7.37 (d, J=8.1 Hz, 1H; ArH), 7.10-6.90 (m, 4H; ArH), 6.45 (s, 1H; N³H), 6.40 (s, 1H; N¹H), 4.50 (dt, J₁=6.5 Hz, J₂=4.8 Hz, 1H; CH), 4.30 (dd, J₁=7.1 Hz, J₂=5.2 Hz, 1H; CH), 4.16 (dd, J₁=6.8 Hz, J₂=3.1 Hz, 1H; CH), 3.15 (dt, J₁=7.5 Hz, J₂=4.9 Hz, 1H; CH), 3.01 (d, J=6.4 Hz, 2H; CH₂), 2.80 (dd, J=4.5 Hz, J_{gem}=12.6 Hz, 1H; CH₂) 2.59 (d, J_{gem}=12.6 Hz, 1H; CH₂), 2.07 (t, J=6.0 Hz, 2H; CH₂), 1.45-1.42 (m, 4H; CH₂), 1.25-1.22 (m, 2H; CH₂).

¹³C NMR (75.5 MHz, DMSO-d₆): 173.98, 172.45, 163.07, 136.38, 127.50, 121.22, 118.66, 111.67, 110.32, 61.2, 59.49, 55.27, 53.11, 40.61, 35.51, 28.28, 27.45, 25.45.

MS (FAB): m/z 431.02 [M+H]⁺.

### Example 8

### Preparation of N-biotinyl-L-norvaline (Gelator 8)

The procedure of Step 1 of Example 1 was repeated, except for employing L-norvaline methyl ester hydrochloride (167 mg, 0.1 mmol) in place of L-phenylalanine methyl ester hydrochloride, increased reaction time of 6 hours, and a mixture of CH₂Cl₂/MeOH (9:1) as an eluant in the column chromatography, to obtain compound 8a (165 mg, yield: 50%). Further, employing compound 8a (179 mg), the procedure of Step 2 of Example 1 was repeated to obtain gelator compound 8 (151 mg, yield: 88%).

M.p. 255° C.

¹H NMR (300 MHz, DMSO-d₆): 8.00 (d, J=7.9 Hz, 1H; NH), 6.39 (s, 1H; N³H), 6.34 (s, 1H; N¹H), 4.28 (dt, J₁=6.5 Hz, J₂=4.8 Hz, 1H; CH), 4.13 (dd, J₁=7.1 Hz, J₂=5.5 Hz, 1H; CH), 4.06 (dd, J₁=6.8 Hz, J₂=3.1 Hz, 1H; CH), 3.04 (dt, J₁=7.5 Hz, J₂=4.9 Hz, 1H; CH), 2.79 (dd, J=4.8 Hz, J_{gem}=13.2 Hz, 1H; CH₂), 2.54 (d, J_{gem}=13.2 Hz, 1H; CH₂), 2.07 (t, J=7.1 Hz, 2H; CH₂), 1.60-1.45 (br, 6H; CH₂), 1.30-1.24 (m, 4H; CH₂), 0.93 (t, J=7.2 Hz, 3H; CH₃).

¹³C NMR (75.5 MHz, DMSO-d₆): 174.34, 172.52, 163.07, 61.32, 59.48, 55.75, 51.67, 38.98, 35.07, 33.34, 28.32, 28.28, 25.58, 19.00, 13.78.

MS (FAB): m/z 343.99 [M+H]⁺.

Anal. Calcd. for C₁₆H₂₇N₃O₄S: C, 52.46; H, 7.34; N, 12.23; S, 9.34Found: C, 52.37; H, 7.46; N, 11.99; S, 9.19.

### Example 9

### Preparation of N-biotinyl-L-norleucine (Gelator 9)

The procedure of Step 1 of Example 1 was repeated, except for employing L-norleucine methyl ester hydrochloride (182 mg, 0.1 mmol) in place of L-phenylalanine methyl ester hydrochloride, to obtain compound 9a (265 mg, yield: 70%). Further, employing compound 9a (186 mg), the procedure of Step 2 of Example 1 was repeated to obtain gelator compound 9 (178 mg).

M.p. 172° C.

¹H NMR (300 MHz, DMSO-d₆): 7.98 (d, J=7.8 Hz, 1H; NH), 6.39 (s, 1H; N³H), 6.33 (s, 1H; N¹H), 4.28 (dt, J₁=7.5 Hz, J₂=5.1 Hz, 1H; CH), 4.14 (dd, J₁=6.1 Hz, J₂=3.7 Hz, 1H; CH), 4.06 (dd, J₁=7.2 Hz, J₂=4.8 Hz, 1H; CH), 3.08 (dt, J₁=5.7 Hz, J₂=4.5 Hz, 1H; CH), 2.80 (dd, J=5.1 Hz, J_{gem}=12.9 Hz, 1H; CH₂), 2.54 (d, J_{gem}=12.9 Hz, 1H; CH₂), 2.10 (t, J=6.0 Hz, 2H; CH₂), 1.62-1.52 (br, 6H; CH₂), 1.80 (m, 4H; CH₂), 1.25 (m, 2H; CH₂), 0.80 (t, J=6.7 Hz, 3H; CH₃).

¹³C NMR (75.5 MHz, DMSO-d₆): 174.02, 172.27, 162.79, 61.09, 59.24, 55.52, 51.67, 40.37, 34.78, 30.75, 28.13, 27.65, 25.31, 21.77, 13.85.

MS (FAB): m/z 358 [M+H]⁺.

Anal. Calcd. for C₁₆H₂₇N₃O₄S: C, 53.76; H, 7.61; N, 11.73; S, 8.97. Found: C, 53.32; H, 7.57; N, 11.60; S, 8.95.

### Example 10

### Preparation of N-biotinyl-D,L-2-aminoenantic acid (Gelator 10)

The procedure of Step 1 of Example 1 was repeated, except for employing D,L-2-aminoenantic acid methyl ester hydrochloride (197 mg, 0.1 mmol) in place of L-phenylalanine methyl ester hydrochloride and increased reaction time of 6 hours, to obtain compound 10a (289 mg, yield: 75%). Further, employing compound 10a (198 mg), the procedure of Step 2 of Example 1 was repeated to obtain gelator compound 10 (194 mg).

M.p. 195° C.

¹H NMR (300 MHz, DMSO-d₆): 7.98 (d, J=7.6 Hz, 1H; NH), 6.34 (s, 1H; N³H), 6.30 (s, 1H; N¹H), 4.24 (dt, J₁=6.7 Hz, J₂=5.0 Hz, 1H; CH), 4.06 (dd, J₁=7.5 Hz, J₂=5.2 Hz, 1H; CH), 4.01 (dd, J₁=6.8 Hz, J₂=4.9 Hz, 1H; CH), 3.01 (dt, J₁=6.9 Hz, J₂=5.1 Hz, 1H; CH), 2.76 (dd, J=4.8 Hz, J_{gem}=12.6 Hz, 1H; CH₂), 2.47 (d, J_{gem}=12.9 Hz, 1H; CH₂), 2.05 (t, J=6.5 Hz, 2H; CH₂), 1.57-1.40 (m, 6H; CH₂), 1.19-1.17 (br, 8H; CH₂), 0.78 (t, J=6.3 Hz, 3H; CH₃).

¹³C NMR (75 MHz, DMSO-d₆): 174.84, 173.10, 163.05, 61.36, 60.05, 56.34, 52.49, 41.18, 35.70, 31.82, 29.03, 28.92, 28.87, 26.16, 25, 94, 22.82, 14.77.

MS (FAB): m/z 372 [M+H]⁺.

Anal. Calcd. for C₁₇H₂₉N₃O₄S: C, 54.94; H, 7.87; N, 11.31; S, 8.63. Found: C, 54.72; H, 7.57; N, 11.40; S, 8.75.

### Example 11

### Preparation of N-biotinyl-D,L-2-aminocaprylic acid (Gelator 11)

The procedure of Step 1 of Example 1 was repeated, except for employing D,L-2-aminocaprylic acid methyl ester hydrochloride (209 mg, 0.1 mmol) in place of L-phenylalanine methyl ester hydrochloride and increased reaction time of 6 hours, to obtain compound 11a (288 mg, yield: 72.1%). Further, employing compound a (200 mg), the procedure of Step 2 of Example 1 was repeated to obtain gelator compound 11 (192 mg).

M.p. 198.5° C.

¹H NMR (300 MHz, DMSO-d₆): 8.12 (d, J=7.5 Hz, 1H; NH), 6.39 (s, 1H; N³H), 6.31 (s, 1H; N¹H), 4.27 (dt, J₁=6.5 Hz, J₂=5.4 Hz, 1H; CH), 4.09 (dd, J₁=7.2 Hz, J₂=4.6 Hz, 1H; CH), 4.00 (dd, J₁=6.4 Hz, J₂=3.5 Hz, 1H; CH), 3.06 (dt, J₁=7.8 Hz, J₂=5.0 Hz, 1H; CH), 2.80 (dd, J=5.8 Hz, J_{gem}=12.6 Hz, 1H; CH₂), 2.54 (d, J_{gem}=12.6 Hz, 1H; CH₂), 2.06 (t, J=6.0 Hz, 2H; CH₂), 1.62-1.42 (m, 6H; CH₂), 1.29-1.18 (br, 10H; CH₂), 0.80 (t, J=5.4 Hz, 3H; CH₃).

¹³C NMR (75 MHz, DMSO-d₆): 174.29, 172.55, 163.05, 61.36, 59.51, 55.80, 51.94, 4.63, 36.53, 31.43, 31.31, 28.54, 28.49, 25.68, 25.62, 22.35, 14.27.

MS (FAB): m/z 386 [M+H]⁺.

Anal. Calcd. for C₁₈H₃₁N₃O₄S: C, 56.08; H, 8.10; N, 10.90; S, 8.32. Found: C, 56.32; H, 7.97; N, 11.04; S, 8.45.

### Test Example 1

### Gelation Capability of Gelators

In order to examine the gelation capability of gelators 1 to 11, the degrees of gelation in various aqueous media were measured as follows, in accordance with the “stable-to-inversion of container” method (Menger, F. M. and Caran, K. L., J. Am. Chem. Soc. 2000, 122, 11679).

Specifically, 0.002 to 0.04 g of each gelator and 1 ml of an aqueous medium (distilled water, 0.9% aqueous NaCl solution, 0.01 M hydrochloric buffer (pH 2.0), 0.05 M phthalate buffer (pH 4.0), 0.08 M MOPSO buffer (pH 7.0) or 0.025 M sodium tetraborate buffer (pH 9.0)) were put into a sealed glass tube (5 mm i.d.), and the mixture was heated at 100° C. until a solution was obtained. The tube was then maintained at room temperature for 5 to 10 min. The resulting sample was considered to be a gel when no phase-separation was visually observed and it did not flow perceptibly upon inversion of the tubes.

Then, the minimum gelation concentration (MGC, wt %), i.e., the lowest concentration of a gelator at which it forms a hydrogel, of each gelator compound was determined, and the result is shown in Table 1.

TABLE 1

| | H₂O/ | | | | |
|---|---|---|---|---|---|
| Gelator | 0.9% NaCl (aq.) | pH 2 | pH 4 | pH 7 | pH 9 |
| 1 | 2.0 | 1.5 | 2.0 | 3.0 | 4.0 |
| 2 | 1.6 | 1.4 | 1.8 | 2.8 | 3.6 |
| 3 | 1.8 | — | — | — | — |
| 4 | 1.2 | 1.0 | 1.2 | 2.7 | 3.9 |
| 5 | 1.4 | 1.0 | 1.4 | 2.7 | 4.0 |
| 6 | — | — | — | — | — |
| 7 | — | — | — | — | — |
| 8 | 1.2 | 0.9 | 1.2 | 2.5 | 3.8 |
| 9 | 0.3 | 0.2 | 0.5 | 1.2 | 1.5 |
| 10 | 0.8 | 0.6 | 1.0 | 2.0 | 4.0 |
| 11 | 0.6 | 0.8 | 0.8 | 1.7 | 3.5 |

—: no gel formed.

As shown in Table 1, gelator 9 exhibited the highest gelation capability, its MGC was 0.3% (8 mM) in distilled water, which means that one molecule of gelator 9 can immobilize 6,700 molecules of water. In an acidic buffer solution at pH 2, the MGC values of the gelators were lower than those in distilled water, and they increased proportionally relative to the pH of the buffer solution.

The gelators having long linear alkyl chains (gelators 9 to 11) showed lower MGC values as compared to the branched amino acid-appended (gelators 2, 4 and 5) and bulky amino acid-appended (gelator 1) gelators.

The β-branched amino acid gelators (gelators 4 and 5) and the short alkyl chain gelator (gelator 8) formed opaque gels which were stable lasting for only about 1 week. In contrast, gelators 1, 2, and 9 to 11 formed very stable gels that persisted for about 6 months in each medium, and the clarities of the gels ranged from translucent to opaque. The MGC values in 0.9% NaCl solution were equivalent to those in distilled water, which suggests that inventive gelator compounds can be used for in vivo applications.

### Test Example 2

### Textures of the Hydrogels Formed by the Gelators

The textures of the hydrogels formed by the gelators prepared in the Examples were examined as follows, employing a scanning electron microscopy (SEM).

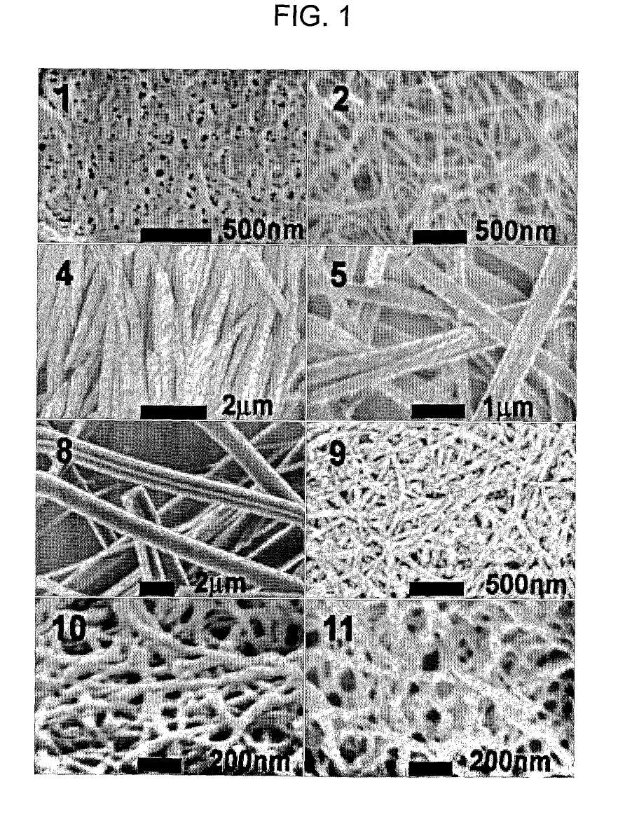

First, gelators 1 to 11 were each dissolved in 1 ml of distilled water in an amount ranging from 0.003 to 0.02 g to reach the corresponding MGC listed in Table 1, and the mixture was heated at 100° C. to form a hydrogel. The hydrogel was frozen at −78° C., and freeze-dried for 6 hours to obtain a xerogel. The resulting xerogels of gelators 1 to 11 were observed with SEM at various magnifications, and the resulting SEM images are shown in FIG. 1.

As shown in FIG. 1, the images of the xerogel revealed two different types of gels, fibrous and lamellar. The hydrogels formed by gelators 1, 2, and 9 to 11 exhibited fibrous structures, wherein the diameter of the fibers ranging from 20 to 50 nm. On the other hand, the hydrogels formed by gelators 4, 5, and 8 showed lamellar structures with higher thickness. The hydrogels having the fibrous microscopic structures were either translucent or opaque, whereas all of the hydrogels having lamellar structures were opaque and less stable.

### Test Example 3

### Mechanism of Self-Assembly of the Gelators

In order to examine the driving forces behind the self-assembly of the gelators, FT-IR and ¹H NMR spectra of the gelators were obtained in accordance with the method of Reference Example, and hydrogen bonding interactions were examined by MOPAC6 modeling.

### (1) FT-IR

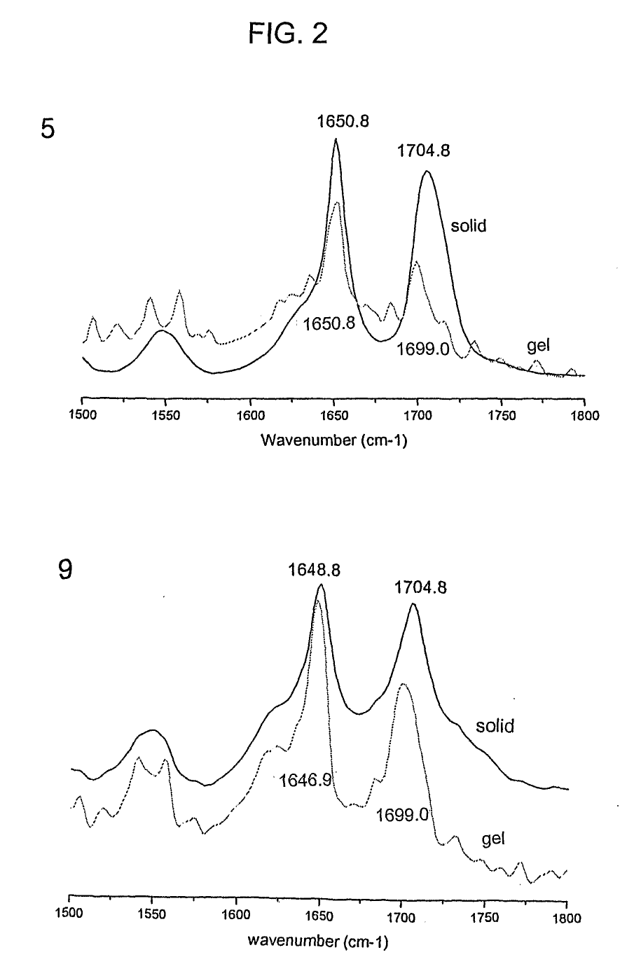

In the FT-IR spectra of gelators 5, 9, and 11 shown in FIG. 2, the carbonyl stretching band of the carboxylic acid moiety of each amino acid unit shifted significantly, to ca. 1699 cm⁻¹, in the gel state relative to its location in the solid amorphous state, i.e., ca. 1705 cm⁻¹. These spectral changes suggest that hydrogen bonding interactions by carboxylic groups provide one of the driving forces for gelation. However, very marginal changes in the C═O stretching frequency of the amide group of the gel state relative to the solid amorphous state (1645-1650 cm⁻¹) indicate that the contribution by amide group towards self-assemble mode is not significant.

### (2) ¹H-NMR

Gelators 1, 2, 4, 5, and 8 to 11 were each dissolved to a concentration of 5 mg/ml in a mixture of DMSO-d₆ and H₂O, having a H₂O content ranging from 0% to 50%, and the changes in the chemical shifts of the protons of the ureido and amido units were examined by ¹H-NMR spectroscopy.

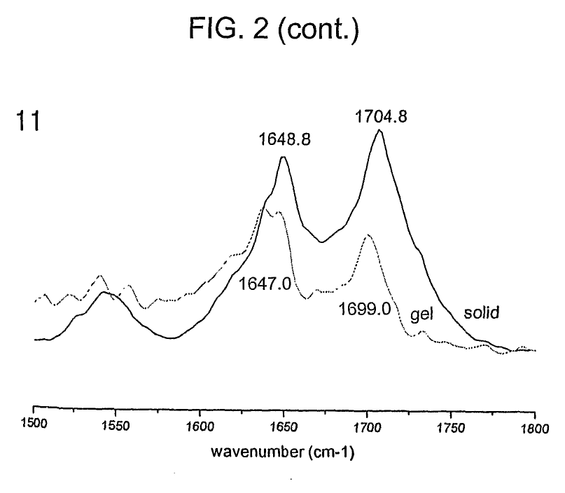

As can be seem from FIG. 3, upon increasing the H₂O content, the amide proton initially shifted to lower field (up to 40% H₂O) and then shifted upfield (>40% H₂O). This observation indicates a change in the nature of the hydrogen bonding, for instance, from (CD₃)₃SO . . . H—N to H₂O . . . H—N (Suzuki, M. et al., Helv. Chim. Acta. 2003, 86, 2228; and Kogigo, M. et al., Chem. Eur. J. 2003, 9, 348). Furthermore, the upfield shifts of the amide NH signal at H₂O concentrations>40% indicate that intermolecular hydrogen bonding occurs among the gelator molecules (Suzuki, M. et al., supra; Kogigo, M. et al., supra; Billiot, F. H. et al., Langmuir 2002, 18, 2993; and Rabenstein, D. L., J. Am. Chem. Soc. 1973, 95, 2797). It has been reported that amide units usually begin to form intermolecular hydrogen bonds at ca. 20-30% H₂O (Suzuki, M. et al., supra). However, in this experiment, the formation of intermolecular hydrogen bonds began at >40% H₂O content. This finding implies that the degree of intermolecular hydrogen bonding of the amide groups is significantly lower than expected, which is consistent with the FT-IR observations. Interestingly, the resonance of the ureido proton began to shift upfield at an H₂O concentration of only 10%. This trend is exactly the same as that observed for biotin itself, which implies that the ureido units form intermolecular hydrogen bondings at this low H₂O concentration and, therefore, they are responsible for the occurrence of the hydrogelation process. In the crystal structure of biotin, the carboxylic acid unit forms a hydrogen bonding with the ureido moiety (DeTitta, G. T. et al., J. Am. Chem. Soc. 1976, 98, 1920) and, accordingly, it is believed that the upfield changes in the chemical shift of the ureido proton are mostly due to intermolecular hydrogen bonding with the carboxylic acid group.

### (3) Hydrogen Bonding Interactions

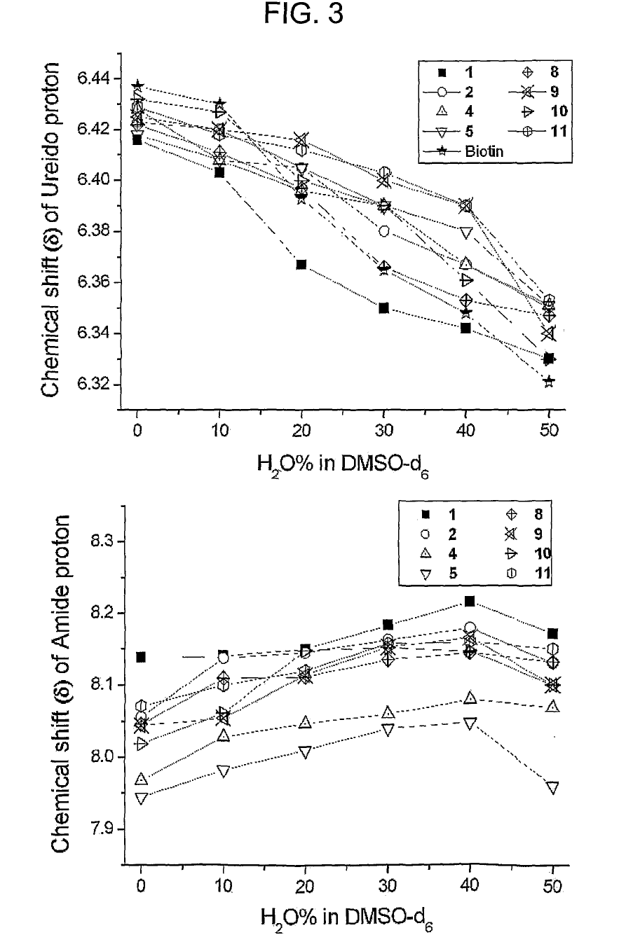

Hydrogen bonding interactions between gelator molecules within a dimer of gelator 5 was examined by employing MOPAC6 software (available at http://www.ccl.net) and the AM1 Hamiltonian with considering the MMOK (molecular mechanics corrections to CONH-types of linkages) (FIG. 4).

As a result, it was found that the most effective hydrogen bonding with lowest heat of formation occurred between the ureido and carboxylic acid groups of the dimer complex. Based on these observations, it can be anticipated that, in biotin-based hydrogelators, the ureido group of the biotin moiety forms an intermolecular hydrogen bonding to the terminal carboxylic acid unit of another gelator molecule, leading to the formation of a self-assembled polymer chain. In addition to these hydrogen bonds, hydrophobic interactions and van der Waals forces play important roles in the gelation process as well as in determining the architectural behavior in the gel state.

### Test Example 4

### Influence of Ligand-Receptor Interaction on a Hydrogel

The influence of ligand-receptor interaction on the gel state of hydrogels was examined as follows.

0.002 g of gelator 9 was added to 1 ml of distilled water, and the mixture was heated at 100° C. to form a hydrogel. 0.002 equivalent of streptavidin was added to the hydrogel and, after 1 hour, the resulting gel was observed with SEM.

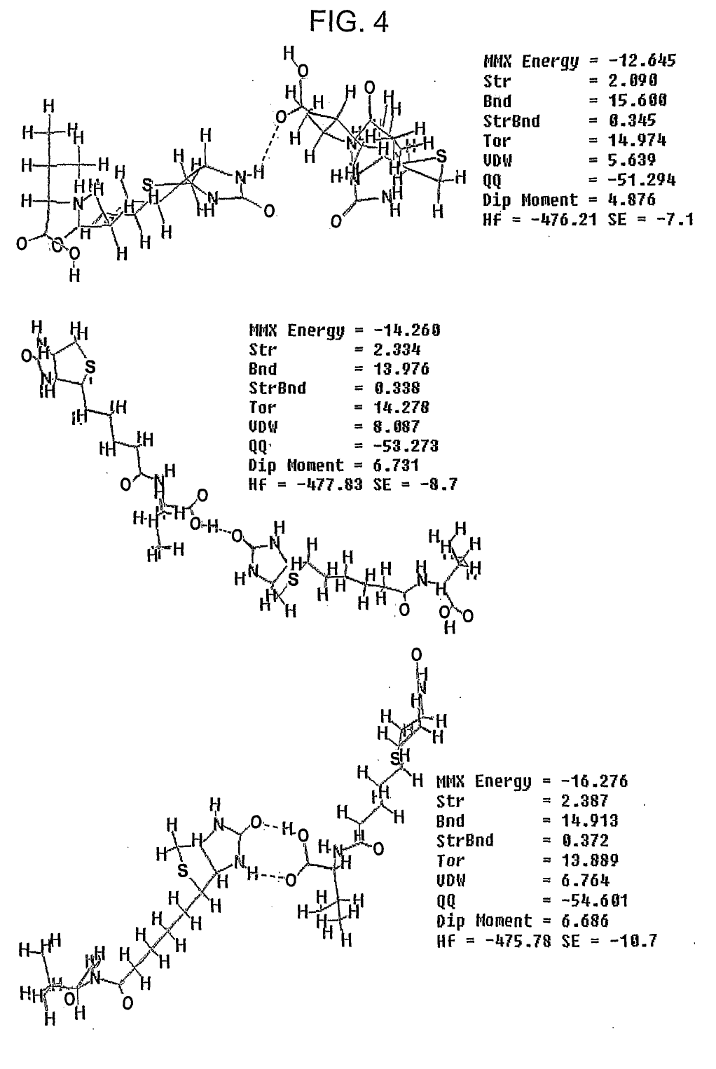

As can be seen from the resulting SEM images of FIG. 5 (a: before the addition of streptavidin; and b: after the addition of streptavidin), the gel state became disrupted due to the addition of streptavidin, and many cracks were observed on the gel fibers of this sample. This result implies that streptavidin molecules specifically bind to the ureido group of biotin by the ligand-receptor interaction, and disrupt the fiber network of the gelator.

### Test Example 5

### Drug Delivery by Biotin-Based Hydrogel

0.3% by weight of gelator 9 was dissolved in 1 ml of 50 μM Zidovudine (AZT) solution or water (blank), and the resulting solution was heated at 10° C. to form a gel. Then, each gel was immersed in 1 ml of water. At given times, the solution was removed from the gel, and UV/VIS absorption of the AZT-containing solution was recorded at 266 nm (λₘₐₓ of AZT) using the blank solution as a reference. After recording, the solution and water were returned to the respective gels. This cyclic process was continued for 9 hours to quantify the time-dependent amount of AZT released from the gel into water.

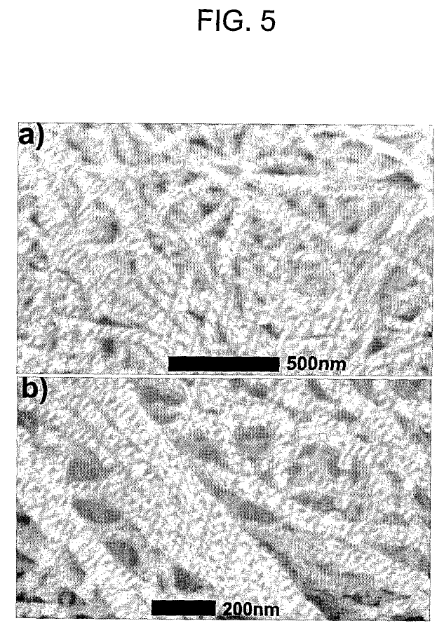

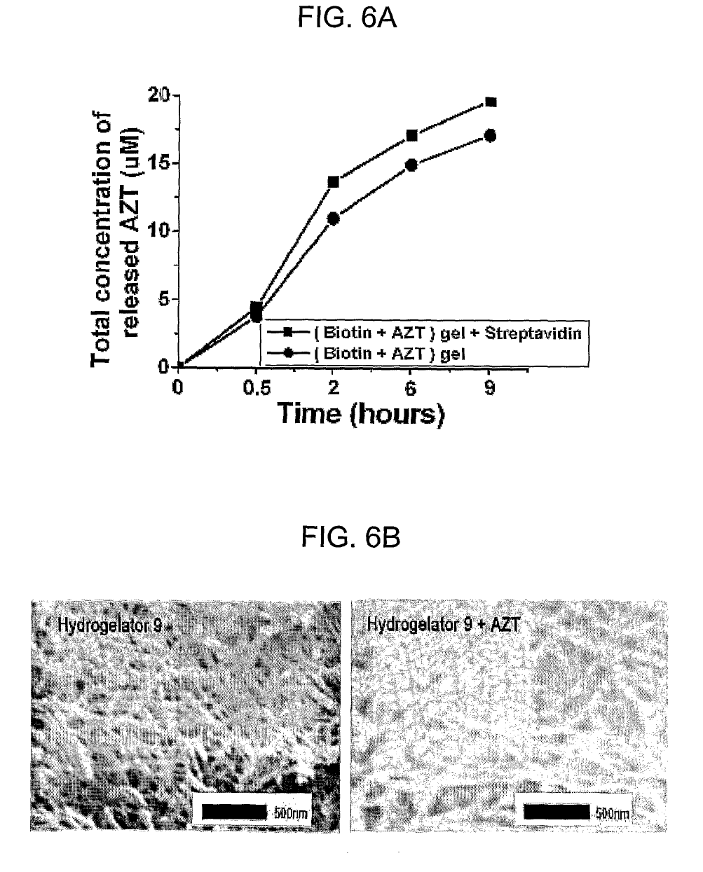

As can be seen from the result of FIG. 6A, only ca. 18% of AZT was released from the gel phase to the water phase after 9 hours and, practically, no UV/VIS absorption was detected for the gel itself.

Further, in order to examine the influence of streptavidin on the AZT release from the hydrogel, the same procedure as above was repeated except for adding 0.002 equivalent of streptavidin to the water in which the gel formed in the AZT solution was immersed. The released AZT amount was observed to increase by about 1 to 7% at each check point (FIG. 6A). These results indicate that drug release from the inventive biotin-based hydrogel can be controlled by employing streptavidin.

Meanwhile, the appearance of the gel was examined before and after incorporating AZT, and it was observed that the gel appearance changed from homogeneous to heterogeneous as it incorporated AZT. The SEM images of the gels before and after AZT incorporation show the change of internal structure (FIG. 6B).

While the invention has been described with respect to the above specific embodiments, it should be recognized that various modifications and changes may be made to the invention by those skilled in the art which also fall within the scope of the invention as defined by the appended claims.

What is claimed is:

1. A biotin-amino acid conjugate, wherein the carboxylic group of biotin and the α-amino group of an amino acid is linked by an amide bond.

2. The biotin-amino acid conjugate of claim 1, which is represented by formula (I): 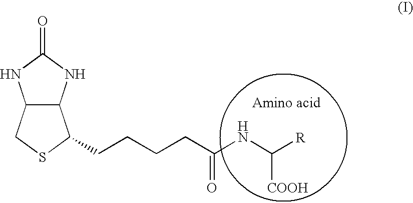
wherein, R is C₁-C₆ alkyl, or C₁-C₃ alkyl substituted with phenyl, methyl, methylthio, hydroxyphenyl or indole.

3. The biotin-amino acid conjugate of claim 2, wherein R is C₄-C₆ alkyl or phenylmethyl.

4. A process for preparing a biotin-amino acid conjugate comprising the steps of:
(a) dissolving D-biotin, 1-(3-dimethylaminopropyl)-3-ethylcarbodiimide.HCl (EDC) and N,N-dimethyl-4-aminopyridin (DMAP) in an organic solvent, adding a methyl ester of an amino acid thereto, and reacting the mixture for 4 to 6 hours to obtain an intermediate compound; and
(b) treating the intermediate compound obtained in (a) with NaOH in a mixture of methanol and distilled water for 4 to 6 hours to obtain the biotin-amino acid conjugate.

5. The method of claim 4, wherein the organic solvent used in step (a) is dimethylformamide.

6. A hydrogel prepared by dissolving the biotin-amino acid conjugate of claim 1 in an aqueous medium.

7. A drug delivery system comprising the hydrogel of claim 6 and a drug incorporated therein.

8. The drug delivery system of claim 7, which further comprises a receptor of biotin.

9. The drug delivery system of claim 8, wherein the receptor of biotin is avidin, streptavidin, cyclodextrin, or insulin.
